(19) United States
(12) Patent Application Publication
(10) Pub. No.: US 2004/0224379 A1
(43) Pub. Date: Nov. 11, 2004

(54) Method and apparatus for detection of toxic substances in a sample

(76) Inventor: Robert L Jaffe, Long Island City, NY (US)

Correspondence Address: OPPEDAHL AND LARSON LLP, P O BOX 5068, DILLON, CO, 80435-5068, US

(21) Appl. No.: 10/258,409

(22) PCT Filed: Apr. 5, 2001
(86) PCT No.: PCT/US01/11079

Related U.S. Application Data
(60) Provisional application No. 60/195,340, filed on Apr. 5, 2000.

(51) Int. Cl. C12Q 1/02; C12Q 1/22

(52) U.S. Cl. 435/29; 435/31; 435/283.1

(57) ABSTRACT

Toxic substances in a sample can be detected by combining the sample with a living culture of Tetramitus rostratus (10), and monitoring (12) the morphological state and/or swimming behavior of the living culture of Tetramitus rostratus in the presence of the sample. Loss of coordination of swimming behavior of the Tetramitus rostratus is indicative of the presence of a toxic substance in the sample. The sample tested may be a solid or liquid sample, or it may be a gaseous sample (for example an air sample (5) which may contain an airborne toxin).

This application claims the benefit of the filing date of U.S. Provisional Application No. 60/195,340, filed Apr. 5, 2000, which is incorporated herein by reference.

### FIELD OF THE INVENTION

This application relates to a method and apparatus for detection of toxic substances in air, water or other solid, liquid or gaseous samples. The method is specifically adapted for use in the context of toxic substances disbursed by terrorist or military opponents, and can be incorporated into field-usable apparatus for use by military and law enforcement personnel. It may also be used in environmental monitoring and in monitoring of food stuffs for contamination.

### BACKGROUND OF THE INVENTION

The challenge of detection of toxic substances which may be dispersed by terrorists or military opponents is substantial. Any such test needs to be robust, rapid, easily interpreted and is preferably sensitive to a variety of toxic substances. The present invention provides a method and an apparatus to meet this challenge.

The method of the present invention makes use of flagellate particle feeding microorganisms. A preferred organism for use in the invention is Tetramitus rostratus in its flagellate form. This organism has been previously shown to exhibit a reduced growth rate and or cell size when exposed to a variety of toxic substances. (See U.S. Pat. No. 5,387,508, which is incorporated herein by reference). However, observation of change in growth rate can be made in periods of time from 2 hours to 24 hours after exposure. Furthermore, it requires fairly sophisticated equipment. Thus, simply observing the growth rate of T. rostratus or similar flagellates does not provide a solution to the challenge of detection of toxic substances which may be dispersed by terrorists or military opponents.

U.S. Pat. No. 6,130,956 discloses monitoring of endogenous microbiota in water samples. Images of are taken to record morphological, color and motion related features of the microorganisms, and these features are evaluated to assess the quality of the water sample. Such a test only evaluates the characteristics of a particular sample, however, and does not make use of a specific exogenous test organism to monitor toxic substances. Furthermore, this test has not disclosed utility for airborne substances.

### SUMMARY OF THE INVENTION

In accordance with the present invention, it has now been found that toxic substances in a sample can be detected by combining the sample with a living culture of Tetramitus rostratus, and monitoring the morphological state and/or swimming behavior of the living culture of Tetramitus rostratus in the presence of the sample. Loss of coordination of swimming behavior of the Tetramitus rostratus is indicative of the presence of a toxic substance or substances in the sample. The sample tested may be a solid or liquid sample, or it may be a gaseous sample (for example an air sample which may contain an airborne toxin).

### BRIEF DESCRIPTION OF THE DRAWINGS

FIG. 1 shows a kill curve for Tetramitus rostratus exposed to spoiled fish;

FIG. 2 shows particle toxicity in samples from 6 reservoirs in the same watershed; and

FIG. 3 shows an embodiment of an apparatus in accordance with the invention adapted for testing of airborne toxic substances.

### DETAILED DESCRIPTION OF THE INVENTION

In addition to the responsiveness of T. rostratus growth rates to the presence of a broad spectrum of toxic substances, it has now been found that T. rostratus possess other characteristics which can be monitored to meet this need, namely morphology and swimming behavior. These changes precede the reduction in growth rate and therefore provide for an earlier indication of the presence of toxic substances.

T. rostratus is a multiflagellate. Because of this, substantial coordination of flagellar motion is required to maintain normal swimming patterns. When T. rostratus is exposed to toxic substances, this coordination is disrupted, leading to abnormal swimming behavior which is observable within 5 to 10 minutes of exposure. This behavior has been observed following exposure to toxins from spoiled fish extract, asbestos, contaminated soil samples and industrial effluents, and is thus apparently a non-specific response which can be used generally to detect toxic substances, including chemical warfare agents.

Table 1. summarizes observed changes in morphology and swimming behavior over time. The most dramatic change is the appearance of cells, which spin around in circles. After 1.6 hours of exposure 5-10% of the cells exposed to 10 μg/mL of 4-nitroquinoline-n-oxide are “spinners”. Early appearance of “spinners” also has been observed where Tetramitus flagellates have been exposed to particle suspensions from the New Croton Reservoir and particle suspensions obtained from contaminated soil samples.

TABLE 1
Microscopic observations of swimming behavior during exposure of
Tetramitus Flagellates to 10 μg/mL of 4-nitroquinoline-n-oxide.

| Exposure | Swimming | Appearance of | Swarming | Spinner |
|---|---|---|---|---|
| Time | Velocity | Larger Morphs⁽¹⁾ | Behavior⁽²⁾ | Frequency⁽³⁾ |
| 0.5 Hours | 10-20% | 1-5% | Normal | <1% |
| | Slow | | | |
| 0.9 Hours | 50-70% | 5-10% | Delayed | ˜5% |
| | Slow | | | |
| 1.6 Hours | 100% | 10-20% | Not | 5-10% |
| | Slow | | complete | |
| 4.5 Hours | 100% | 50% | Not | 20% |
| | Slow | | complete, | |
| | | | slow | |
| 8.7 Hours | Very Slow | 100% Rounded cells | No | 70-90% |
| | | with large vacuoles | Swarming | |

1—Larger Morphs—Larger cells with abnormally-formed rostrum and large vacuoles. These cells are unable to complete cell division and are presumed to decrease in size with time.

2—Swarming Behavior—Flagellates form a dense concentration of cells around the perimeter of the coverslip-chamber. The chamber can be either a regular microscope slide with a 22 mm square coverslip with small clay feet corners, or the standard hemacytometer/coverslip configuration.

3—Spinner cells rotate in a circular pattern as contrasted to the straight-ahead direction of non-exposed flagellates.

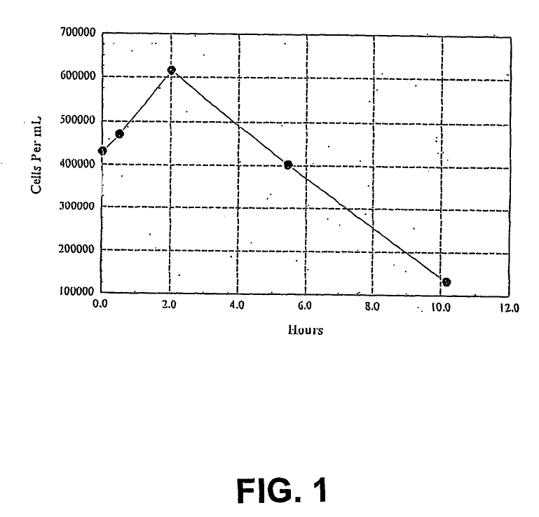

Exposure of Tetramitus flagellates to a buffered (MS-1) extract of spoiled fish resulted in a dramatic decrease in the mean cell diameters after 2 hours of incubation (Table 2). Starting with two hours a linear kill curve also was observed (FIG. 1). Although decrease in mean cell diameters is the general rule for exposure to soluble toxic agents, the effect of chrysotile asbestos presents a different picture. Starting with 4 hours of incubation there is a linear increase in the number of large fan-like cells. These cells result from the failure of the mitotic spindles to separate as a result of exposure to the chrysotile asbestos and represent double-sized cells frozen at this stage of mitosis.

TABLE 2
Mean cell diameter of Tetramitus flagellates after incubation with a
50% extract of “Spoiled” Turbot. Mean cell diameters were determined
with the Accucomp ™ program of the Coulter Multisizer IIe.

| Incubation Time | Mean Cell Diameter | |
|---|---|---|
| (Hours) | (μ) | Cells Per mL |
| 0.03 | 9.53 | 4.30 × 10⁵ |
| 0.51 | 9.41 | 4.70 × 10⁵ |
| 2.02 | 8.34 | 6.16 × 10⁵ |
| 5.45 | 8.42 | 4.02 × 10⁵ |
| 10.18 | 5.56 | 1.32 × 10⁵ |

Applying these observations in a real-world context, a first aspect of the present invention is a method for the detection of a toxic substance, if present, in an environmental sample, such as air or water. The method can also be used to monitor purity of foodstuffs, to detect toxic substances in biological fluids such as urine, and for a rapid test for detection of toxic substances in industrial effluents. In accordance with the method of the invention, a sample suspected of containing a toxic substance is added to a culture of live T. rostratus in flagellate form. The morphology and/or swimming behavior of the culture after the addition of the sample is compared to the morphology and/or swimming behavior before the addition of the sample, or to a standard morphology or swimming behavior pattern. Meaningful changes in morphology and/or swimming behavior can frequently be observed within 5-10 minutes of the addition of the sample, although monitoring for longer periods of time, for example up to an hour, would still produce useful results.

Monitoring of morphology can be carried out by microscopic examination of the T. rostratus after combination with the sample. Monitoring of swimming behavior can be performed visually by microscopic evaluation. Prior to exposure to a toxic substances, T. rostratus tend to swim in a straight line. This is particularly true when one uses oxygen to stimulate a chemotactic migration towards the surface of the culture medium. In contrast, after exposure to a toxic substance, T. rostratus lose flagellar coordination and tend to swim in circles.

The observation of changes in swimming behavior could also be automated. Thus, for example, software which maps and characterizes object motion patterns can be utilized to analyze observed motion patterns and compare them to standards (stored reference standards or pre-exposure standards from the same culture) to detect a change to abnormal swimming behavior.

Measurement of unconcentrated (neat) samples can be achieved with a continuous injection method. Steady-state cultures of Tetramitus flagellates are maintained in a 1000 mL spinner flask (volume and size of flask can be varied to permit different rate/concentrations of sample injection). The concentration of cells in the population is maintained at a constant level (1×10⁵-1×10⁶ cells per mL) by regulation of the flow of nutrient media and washout volumes. Cell number and cell size are monitored by a double beam laser sensor. Continuous flow injection of culture effluent into the flow-through cell of the laser sensor is commercially available. The signal from the sensor is analyzed with a software program, which feeds back into a controller which regulates the volume of nutrient and washout rate in the spinner flask. This method regulates the cell concentration. At specific toxicant levels, changes in cell volume will occur. The software program of the laser sensor can parse the cell size distributions into subpopulations of specific size ranges. Overlays of size patterns from exposed cultures can be superimposed over the “normal” distributions and % differentials can be calculated. Alarm thresholds can be established to warn of toxicant levels exceeding specified values. Confirmation of toxicity by measuring cell division inhibition in regular shaking cultures can be used to confirm the validity of the “early warning measurements”. This method has a range of applications from monitoring water purification processes to the production of various liquid products. At very high toxic levels cell death may occur and decrease in the cell concentrations will be observed.

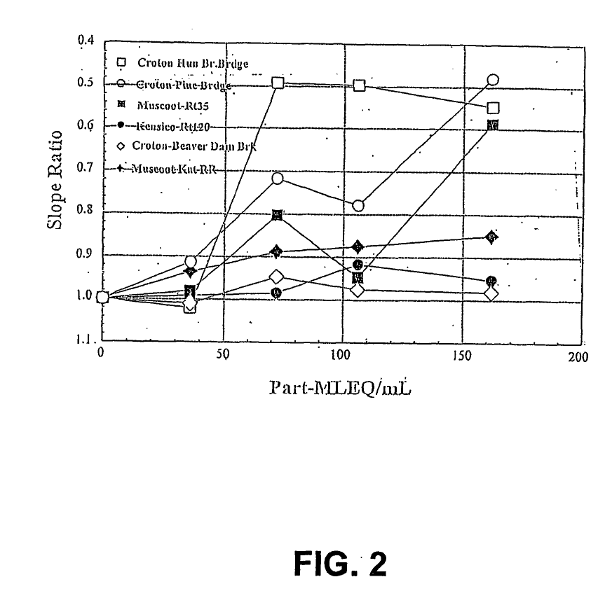

Measurements of Tetramitus toxicity in samples, taken from reservoirs in the Croton Watershed revealed no toxicity (cell division inhibition) in the unconcentrated (neat) samples. However, when particle suspensions were prepared by centrifugation and resuspension of the particles in smaller volumes, toxicity was observed (FIG. 2).

Particle suspensions can be diluted and parsed into the wells of microtitre plates containing growing cultures of flagellates. The cell concentrations and cell size distributions can be measured by transfer of aliquots to the flow-through cells of the laser sensor. An auto sampler with a programmed wash cycle and sequential solenoid control of delivery of aliquots from the microtitre plates permits automated monitoring of each incubate. The concentration of particles is expressed as Particle-MLEQ/mL (particle milliliter equivalents per mL). Thus 50 Part-MLEQ/mL is the number of particles in the particle suspension which were present in 50 mL of the unconcentrated sample. Particle counts in both neat sample and the particle suspension preparations are counted in a ZM Coulter Counter or the laser particle counter in order to evaluate the recovery after centrifugation. Sequential filtration through a graded series of filters of known pore size allows for the preparation of subpopulations of particles of known size ranges (i,e., 10-20μ, 5-10μ, 1-5μ, etc.). The size fractionation of particle subpopulations is useful in tracking components of heterogeneous particle mixtures back upstream in order to determine their points of origin. Thus, a semi-automated monitoring procedure is achieved for measuring concentrated particle suspensions.

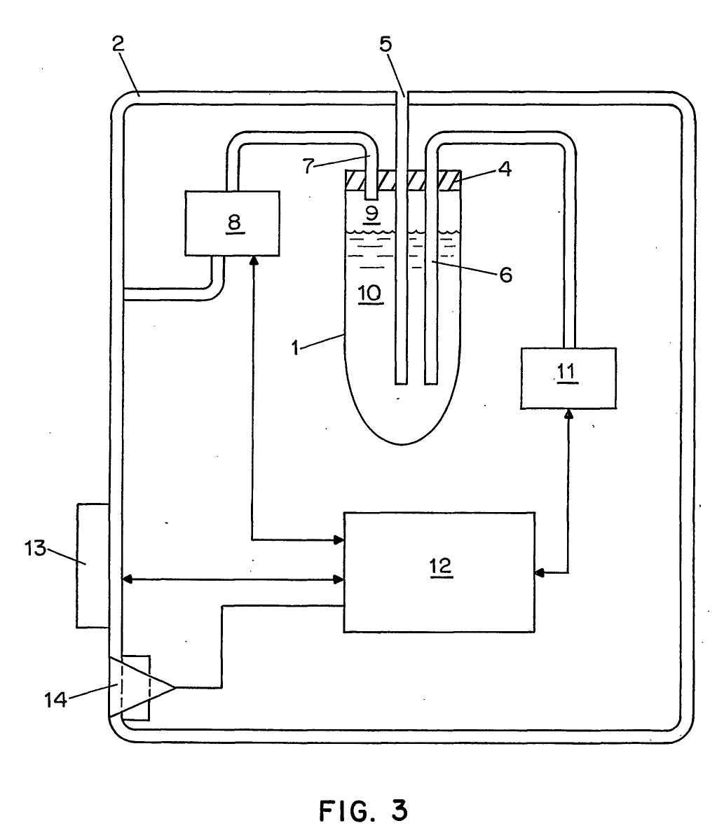

The method of the invention is suitably implemented in an apparatus for providing early warning signals of the presence of toxic substances. Such an apparatus is easy to use, and does not require special training for the interpretation of the results. FIG. 3 shows an embodiment of an apparatus in accordance with the invention adapted for testing of airborne toxic substances. A container 1 of T. rostratus culture 10 is disposed within a housing 2. The container has a stopper 4 through which pass a sample inlet tube 5 which is in communication with the exterior of the housing 2; an analysis probe 6 and a vent tube 7. The vent tube 7 is connected to a pump 8 which draws air out of the space 9 in the container 1 above the T. rostratus culture 10, thus causing air to be drawn in through the sample inlet tube 5. The analysis probe 6 includes an optical fiber and a magnifier for observation of T. rostratus within the culture. The probe may also include a light source, although this may also be disposed external to the tube. The optical fiber is connected to an imaging system 11, such as a CCD array for the capture of images of T. rostratus swimming behavior.

The pump 8 and the imaging system 11 are connected to a processor 12. The processor 12 is also connected to an I/O interface 13 and an audible alarm 14. When the system is activated by inputting a command on the I/O interface 13, the processor 12 sends a signal to start the pump 8 which starts drawing external air into the culture 10 via the sample inlet tube 5. The imaging system starts sending images of the swimming behavior of the T. rostratus to the processor which evaluates the observed patterns in comparison to a standard (either one based on the actual culture or a stored reference). If the swimming behavior is identified as “abnormal”, a signal is is transmitted by the processor 12 to sound the audible alarm 14.

To maximize the ability to assess swimming behavior, it may be desirable to stop the pump 8 for a period of time prior to the taking of a measurement, so that the sample is quiescent and not being stirred by the aerating effects of the sample intake. For non-gaseous samples, other means for stirring the culture may be employed, including magnetic stirrers or a shaking platform. In addition, it may be desirable to take the observations at a defined time following an external stimulus, since this may facilitate data interpretation. Thus, for example, because T. rostratus is attracted to oxygen, the organisms will all tend to swim in one direction towards the surface of the culture if there is no impairment of the flagellar coordination. In the presence of a toxic substance that impairs coordination, however, the ability to swim in this direction will be impaired, and an image will show far more random orientation. Thus, by pulsing the pump or otherwise stirring the same to randomly distribute the T. tetramitus and then taking of an observation of the orientations of the organisms shortly thereafter one can distinguish between a normal culture in which there is an orientation towards the surface, and a culture that has been exposed to a toxic substance.

Field cultures of Tetramitus flagellates may be easily maintained by serial transfer of small volume aliquots from a growing 2 mL, tube-culture to a new culture tube containing fresh medium. The transfer tube with fresh medium can be prepared at a field station and used for upwards of three weeks in the field. Thus, the apparatus of FIG. 3 can be used in the field over extended periods.

Apparatus similar to that shown in FIG. 3 could also be used for the monitoring of industrial effluents and ground water samples to allow continuous or periodic monitoring of such samples for the presence of toxic substances.

1. A method for detection of toxic substances in a sample, comprising the steps of:
(a) combining the sample with a living culture of Tetramitus rostratus in flagellate form, and
(b)) monitoring the swimming behavior of the living culture of Tetramitus rostratus in the presence of the sample, wherein loss of coordination of swimming behavior of the Tetramitus rostratus is indicative of the presence of a toxic substance in the sample.

2. The method of claim 1, wherein the sample is a liquid sample.

3. The method of claim 1, wherein the sample is a gaseous sample.

4. The method of claim 1, wherein the swimming behavior of Tetramitus rostratus is monitored by capturing an image of the Tetramitus rostratus in the presence of a sample.

5. The method of claim 4, wherein the image is captured using an optical fiber and an imaging system.

6. The method of claim 5, wherein the imaging system comprises a CCD array.

7. An apparatus for assessing a sample for the presence of toxic substances, comprising:
(a) a housing;
(b) a reservoir disposed within the housing, said reservoir containing a living culture of Tetramitus rostratus in flagellate form;
(c) a sample inlet tube, passing through the housing to allow introduction of sample from outside the housing into the reservoir;
(d) an analysis probe comprising means for observing the swimming behavior of Tetramitus rostratus in the reservoir; and
(e) means for periodically stirring the Tetramitus rostratus in the reservoir.

8. The apparatus of claim 7, wherein the analysis probe comprises an optical fiber and an imaging system.

9. The apparatus of claim 8, wherein the imaging system comprises a CCD array.

10. The apparatus of any of claims 7-9, wherein the means for periodically stirring the Tetramitus rostratus in the reservoir comprises a pump for injecting a gas into the culture within the reservoir.

11. The apparatus of any of claims 7-9, further comprising a vent tube and a pump, said pump drawing a gaseous sample in through the sample inlet tube mid expelling gas through the vent tube.

12. The apparatus of claim 11, wherein the pump also serves as the means for periodically stirring the Tetramitus rostratus in the reservoir.
